(19) United States
(12) Patent Application Publication
(10) Pub. No.: US 2004/0082847 A1
(43) Pub. Date: Apr. 29, 2004

(54) System and methods for identifying brain regions supporting language

(76) Inventor: Kathleen B. McDermott, St. Louis, MO (US)

Correspondence Address: HARNESS, DICKEY, & PIERCE, P.L.C, 7700 BONHOMME, STE 400, ST. LOUIS, MO, 63105, US

(21) Appl. No.: 10/692,157

(22) Filed: Oct. 21, 2003

Related U.S. Application Data
(60) Provisional application No. 60/420,799, filed on Oct. 23, 2002.
(60) Provisional application No. 60/429,603, filed on Nov. 27, 2002.

(51) Int. Cl. A61B 5/05

(52) U.S. Cl. 600/410

(57) ABSTRACT

A method of identifying one or more language regions in the brain of a subject. The method includes presenting to the subject one or more lists of related words to selectively challenge one or more language systems of the brain, and scanning the brain while presenting the one or more lists. This non-surgical method can be used to obtain language maps for patients awaiting neurosurgery.

### CROSS-REFERENCE TO RELATED APPLICATIONS

This application claims the benefit of U.S. Provisional Application No. 60/420,799, filed on Oct. 23, 2002 and U.S. Provisional Application No. 60/429,603, filed on Nov. 27, 2002. The disclosures of the above applications are incorporated herein by reference.

### FIELD OF THE INVENTION

The present invention relates generally to cerebral assessment procedures and, more particularly, to a method of identifying language regions in the brain of a person.

### BACKGROUND OF THE INVENTION

Neurosurgical procedures for treating patients with such conditions as intractable seizures or brain tumors in left frontal and temporal cortices often require localization of language function. A neurosurgeon attempts to identify the brain regions supporting language for an individual patient, so that these regions can be spared in surgery. In one commonly used method for discovering speech centers of the brain, for example, as part of a strategy for removing tumor material, a neurosurgeon opens the cranium of a patient and electrically stimulates areas of the brain while the patient is awake. The patient is expected to answer questions from the surgeon during the open-cranium mapping procedure.

Intraoperative cortical stimulation mapping can identify regions responsible for language function, but such procedures require a patient to be awake for a portion of the surgical procedure. Substantial effort also is required on the part of the patient, who typically is asked to name a series of pictures during the surgery. These procedures are time consuming. More importantly, however, these procedures cannot divulge before surgery where language resides in the brain of a patient. Only after surgery has been initiated can the surgeon determine whether a region is inoperable due to its recruitment in language function. When a language area and a tumor are co-localized or adjacent to one another, the surgeon may elect not to remove the tumor. In such cases, the patient has undergone a burdensome surgery in which a diagnosis may have been accomplished, but not the ultimate surgical goal.

A second method, known as the Wada technique, can be performed before surgery. This non-surgical technique can suggest whether a patient's language sites reside mostly on the left or right hemisphere of the brain. When surgery is being performed in a hemisphere in which language resides, it is preferable to have specific information as to which hemispheric regions should be spared (due to their importance in language function). The Wada technique, however, does not show specifically where in a hemisphere a language site resides. Additionally, some individuals have bilateral language regions, i.e., language regions on both the left and right side of the brain. The Wada technique can show inconclusive results for such patients.

Whenever possible, neurosurgeons strive to spare cortical sites that are critical for language function. It can be seen, however, that the previously described techniques for locating language function in individual patients have drawbacks. It would be desirable to use functional magnetic resonance imaging (fMRI) for pre-operative language area mapping, so that surgical electrical stimulation mapping might be avoided. It has been thought that functional MRI might be used to localize language areas more precisely than, for example, the Wada technique. Protocols have been attempted, for example, in which subjects are presented with words every one or two seconds while being scanned and are asked to press a button upon making a decision about a word. Such protocols, however, have not elicited robust signals within individual subjects.

### SUMMARY OF THE INVENTION

The present invention, in one embodiment, is directed to a method of identifying one or more language regions in the brain of a subject. The method includes presenting to the subject one or more lists of related words to selectively challenge one or more language systems of the brain, and scanning the brain while presenting the one or more lists.

### BRIEF DESCRIPTION OF THE DRAWINGS

The present invention will become more fully understood from the detailed description and the accompanying drawings, wherein:

FIG. 1 is a view of a subject being scanned in accordance with one embodiment of the present invention;

FIG. 2 illustrates views of left hemispheric regions indicating results obtained using an embodiment of a method of identifying language regions;

FIG. 3 illustrates views of right hemispheric regions indicating results obtained using an embodiment of a method of identifying language regions;

FIG. 4 illustrates contrasts between attention to semantics and to phonology for individual subjects, obtained using an embodiment of a method of identifying language regions; and

FIG. 5 illustrates two-dimensional, flattened representations of cortical regions emerging from contrasts, obtained using an embodiment of a method of identifying language regions.

### DETAILED DESCRIPTION OF EXEMPLARY EMBODIMENTS

It should be understood that the detailed description and specific examples, while indicating certain embodiments of the invention, are intended for purposes of illustration only and are not intended to limit the scope of the invention.

The following description of embodiments of the present invention is merely exemplary in nature and is in no way intended to limit the invention, its application, or uses. Although embodiments of the present invention are described herein in connection with brain surgery, the invention is not so limited. Embodiments of the invention can be practiced in a variety of surgical and non-surgical environments in which it may be desirable to locate brain regions that support language.

The invention, in one embodiment, is directed to a method of identifying one or more language regions within the brain of a subject, including but not limited to a medical patient. One or more word lists are designed to selectively challenge a cortical language system. Short word lists (for example, sixteen words in a list), including, for example, semantically related words (such as “bed” and “rest”) or rhyming words (such as “weep” and “beep”) are presented rapidly to the patient. Words are presented rapidly, for example, at about 560 milliseconds per word. There can be, for example, an approximately 50-millisecond gap between words. The list is presented to the subject while the brain of the subject is being scanned. In the present embodiment, a fast-blocked design with functional magnetic resonance imaging (fMRI) is used. The patient is asked to try to pay attention to relations among the words. Before a list is given to the patient, the patient is given a cue that is instructive as to how words in the list will be related to one another. For example, where a list includes words such as “bed” and “rest”, a cue could be “meaning”. As another example, where a list includes words such as “weep” and “beep”, a cue would be “rhyme”. Rapidity of word presentation can vary. For example, in embodiments used in relation to children, or in relation to individuals having lower than normal verbal IQ, word presentation may be slower than previously described.

In one embodiment, a plurality of word lists are designed to serve as stimuli to the subject. A median word length is, for example, five letters, although other median word lengths could be used. Median word frequencies may be, for example, 23 per million for a semantic list and 13.5 per million for a phonological list. The words are presented rapidly so as to be comprehensible but challenging to the subject.

In one embodiment, a rapidly alternating blocked design is used for stimulus presentation with functional magnetic resonance imaging. A “rapidly alternating” blocked design includes a blocked design in which one or more lists of semantically related words are alternated with one or more lists of phonologically related words. Other ways and/or sequences of designing and/or presenting one or more lists could be used in other embodiments.

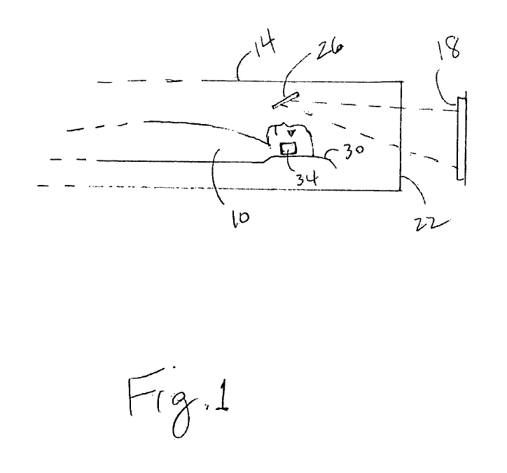

FIG. 1 illustrates a subject being scanned according to one embodiment of the present invention. The subject 10 undergoes functional MRI in a scanner 14. Stimuli are displayed on a screen 18 placed at the head of the bore 22 of the scanner. The subject views the screen 18 via a mirror 26 fastened to a head coil (not shown) of the scanner 14. A pillow 30 and surgical tape minimize head movement. Headphones 34 can dampen scanner noise and can allow communication with the subject.

A blocked design can be used, for example, such that the subject studies semantic and phonological lists (randomly-ordered) within a run. In one embodiment, at the beginning of each block (i.e. list), a cue is displayed (e.g., “meaning” or “rhyme” as previously described) to inform the subject as to a type of list about to be presented, and the subject is instructed to use the cue to help him or her focus on relations among the upcoming words. Words are displayed rapidly, such that, for example, a 16-word list is displayed in about ten seconds. Words are displayed one at a time, for example, for approximately 560 milliseconds apiece with an inter-stimulus interval of approximately 50 milliseconds. In one embodiment, presentation of a block of words is followed by a brief period (for example, about 12.5 seconds), in which the subject is shown, for example, a crosshair and asked to fixate on it and await another list.

A subject is instructed to attend closely to the relations among words within a list. For example, in the semantic condition, the subject is told to think about how the words could be meaningfully connected (e.g. “tiger”, “circus”, “jungle”), and in the rhyme condition the subject is told to think about how the words sound alike (e.g. “skill”, “fill”, “hill”) and to think or say the words silently to himself or herself while thinking about the similarity in the sounds.

In one embodiment, scans are obtained on the scanner 14 using a circularly-polarized head coil. A word list is displayed using a computer (not shown) and appropriate software. A list is displayed on the screen 18. (Alternative scanning and computing equipment and software could be used in other embodiments.) The subject views the screen 18 via the mirror 26.

Structural images are acquired, for example, using a high-resolution sagittal MPRAGE sequence (1.25 mm×1 mm×1 mm voxels). Functional images are collected, for example, with an asymmetric spin-echo-planar sequence sensitive to blood-oxygenation-level-dependent (BOLD) contrast. In a functional run, for example, 128 sets of 16 contiguous, 8 mm-thick axial images (TR=2500 ms, 3.75 mm×3.75 mm in-plane resolution) are acquired parallel to the anterior-posterior commissure plane.

A blocked design can be used, in which onset of lists coincide with onset of a TR (repetition time). Each task block can span, for example, five TRs: an orienting word or cue can appear for about 2 seconds, followed by words in the list.

An exemplary method shall be described in which functional magnetic resonance imaging (fMRI) techniques were used to identify neural regions associated with attention to semantic and phonological aspects of written words within a group of subjects. Short lists (for example, sixteen words per list) including visually-presented semantically-related words (e.g., “bed” and “rest”) or rhyming words (e.g., “weep” and “beep”) were presented rapidly to the subjects, who were asked to attend to relations among the words. Regions preferentially involved in attention to semantic relations appeared within left anterior/ventral inferior frontal gyrus (IFG, approximate Brodmann Area, BA47), left posterior/dorsal IFG (BA44/45), left superior/middle temporal cortex (BA22/21), left fusiform gyrus (BA37), and right cerebellum. Regions preferentially involved in attention to phonological relations appeared within left inferior frontal cortex (near BA6/44, posterior to the semantic regions within IFG described above) and within bilateral inferior parietal cortex (BA40) and precuneus (BA7). This method is notable in that a comparison of the two tasks within some of the individual subjects revealed activation patterns similar to the group average, especially within left inferior frontal and left superior/middle temporal cortices. This fact, combined with the efficiency with which the data can be obtained (for example, in about one hour of functional scanning) and the adaptability of the task for many different subject populations, suggests a wide range of possibilities for embodiments of the present invention. For example, embodiments could be used to track language development (e.g., in children), compare language organization across subject populations (e.g., for dyslexic or blind subjects), and identify language regions within individuals (e.g., to aid in surgical planning).

Two broad classes of processes implicated in single-word reading are semantic (or meaning-based) processing and phonological (or sound-based) processing. It has been demonstrated that false memories can be created by challenging semantic and phonological systems. When presented with semantic associates, people often later recall and recognize having heard a word related to the presented associates but not itself presented. For example, after encountering “bed, rest, awake, tired . . . ”, people may recall and recognize having studied “sleep”. Similarly, phonologically-related words can lead to false memories; after studying “sweep, steep, sleet, slop”, people may mistakenly recall and recognize “sleep”.

In one embodiment of the present invention, logic used in creating false memory paradigms is applied to study language; that is, lists of associated words are used to separately challenge semantic and phonological systems in order to pull apart regions preferentially activated for semantic and phonological processing. Thus embodiments of the present invention can serve, for example, as a tool with which to identify regions differentially activated by attention to semantics and to phonology.

### EXAMPLE

Subjects (N=20, 18 females, mean age 22.1, range 18-32 years) were recruited. All reported being right-handed native speakers of English with normal or corrected-to-normal vision and no history of significant neurological problems.

Seventy-two word lists served as stimuli. Lists included sixteen words related to one another semantically (e.g. “bed”, “rest”, “awake”) or phonologically (e.g. “weep”, “beep”, “heap”). The phonologically-related words all rhymed. The median word length was five letters for both the semantic and phonological lists, and the median word frequency was 23 per million for the semantic lists and 13.5 per million for the phonological lists.

In six encoding runs, subjects studied seventy-two 16-word lists (12 lists per run). A blocked design was used, such that each subject studied semantic and phonological lists (randomly-ordered) within each run. At the beginning of each block (i.e. list), a cue was displayed (“meaning” or “rhyme”) to inform subjects of the type of list they were about to see, and they were instructed to use the cue to help them focus on the relations among the upcoming words. Words were displayed rapidly, such that each 16-word list was displayed in 10 seconds. Words were displayed one at a time for approximately 560 milliseconds apiece with a 50-millisecond interstimulus interval. Following each block of words was a brief period (12.5 seconds), in which subjects were shown a crosshair and asked to fixate on it and await the next list.

Subjects were instructed to attend closely to the relations among words within each list. In the semantic condition, they were told to think about how the words could be meaningfully connected (e.g. “tiger”, “circus”, “jungle”), and in the rhyme condition they were told to think about how the words sounded alike (e.g. “skill”, “fill”, “hill”) and to say the words silently to themselves while thinking about the similarity in the sounds. Subjects were informed that memory tests would occur after some runs but that they should simply focus on the task at hand while viewing the lists.

In the present example, scans were obtained on a 1.5 Tesla Vision System by Siemens, of Erlangen, Germany using a standard circularly-polarized head coil. Visual stimuli were displayed using a Power Macintosh computer by Apple, Cupertino, Calif. and Psyscope software. Psyscope is described in Cohen, J. D., et al., Psyscope: A New Graphic Interactive Environment For Designing Psychology Experiments, Behavior Research Methods, Instruments & Computers 1993; 25:257-71. A liquid crystal display (LCD) projector shielded with copper wire displayed stimuli on a screen placed at the head of the bore of the scanner. Subjects viewed the screen via a mirror fastened to the scanner head coil. A pillow and surgical tape minimized head movement. Headphones dampened scanner noise and allowed communication with subjects.

Structural images were acquired using a high-resolution sagittal MPRAGE sequence (1.25 mm×1 mm×1 mm voxels). Functional images were collected with an asymmetric spin-echo-planar sequence sensitive to blood-oxygenation-level-dependent (BOLD) contrast. In each functional run, 128 sets of 16 contiguous, 8 mm-thick axial images (TR=2500 ms, 3.75 mm×3.75 mm in-plane resolution) were acquired parallel to the anterior-posterior commissure plane; this procedure offered whole-brain coverage at a high signal-to-noise ratio. Approximately 3 minutes elapsed between runs, during which time instructions were given to subjects over their headphones. The first four images of each run were not included in the functional analyses but were used to facilitate alignment of the functional data to the structural images.

Each subject participated in six runs. After each of the first three runs, a recognition memory test was administered. A blocked design was used. Onset of lists coincided with onset of a TR (repetition time). Each task block spanned five TRs: the orienting word appeared for 2 seconds, followed by the 16 words in the list. Ordering of the blocks was unpredictable from the subjects' standpoint.

Data for each subject were corrected for intensity differences across odd- and even-numbered slices, interpolated to 3 mm×3 mm×3 mm voxels, aligned to correct for slice-based within-trial differences in acquisition times, movement-corrected within and across runs, and transformed into standardized atlas space via a linear warp. Removal of the linear slope on a voxel-by-voxel basis corrected for frequency drift, whole brain normalization to a common mode of 1000 facilitated comparisons across subjects, and a Gaussian smoothing filter (6 mm full-width half-maximum) accommodated variations in activation loci across subjects.

Similarities between activation during semantic and phonological lists were demonstrated qualitatively by performing separate random effects t-tests for each 3 mm isotropic voxel on the activation magnitudes (percent signal change) for the semantic lists relative to the control period and for the phonological lists relative to the control period. That is, activation magnitude estimates were obtained for each voxel for each subject for each condition (semantic list, phonological list, and control period); dependent-measures t-tests were then performed for each voxel for the semantic control contrast and for the phonological-control contrast. Regions demonstrating preferential activation for one type of list over the other were obtained using a similar t-test on the activation magnitudes for the semantic and phonological lists for each 3 mm isotropic voxel.

It was decided that to achieve a whole-brain P-value of 0.05, only voxels exceeding P<0.0012 that were also contiguous with at least 11 other voxels exceeding this threshold would be accepted.

An automated peak-search algorithm was applied to the multiple-comparison corrected image resulting from the semantic-phonological t-test to identify the location (in atlas coordinates) of peak activations on the basis of level of statistical significance. Regions around the peak activations were identified interactively by choosing contiguous voxels surpassing the significance threshold.

The statistical activation maps in Talairach and Tournoux atlas space were displayed using the Computerized Anatomical Reconstruction and Editing Toolkit (CARET) software, which is obtainable at http://stp.wustl.edu. See Van Essen, et al., An Integrated Software Suite For Surface-Based Analyses of Cerebral Cortex, Journal of the American Medical Informatics Association 2001;8:443-59. This software was used to view cortical activations projected onto the surface of a high resolution structural brain image and to flatten the cortical data for display in two dimensional “flatmaps” to enable views of the entire left and right hemispheres within one figure.

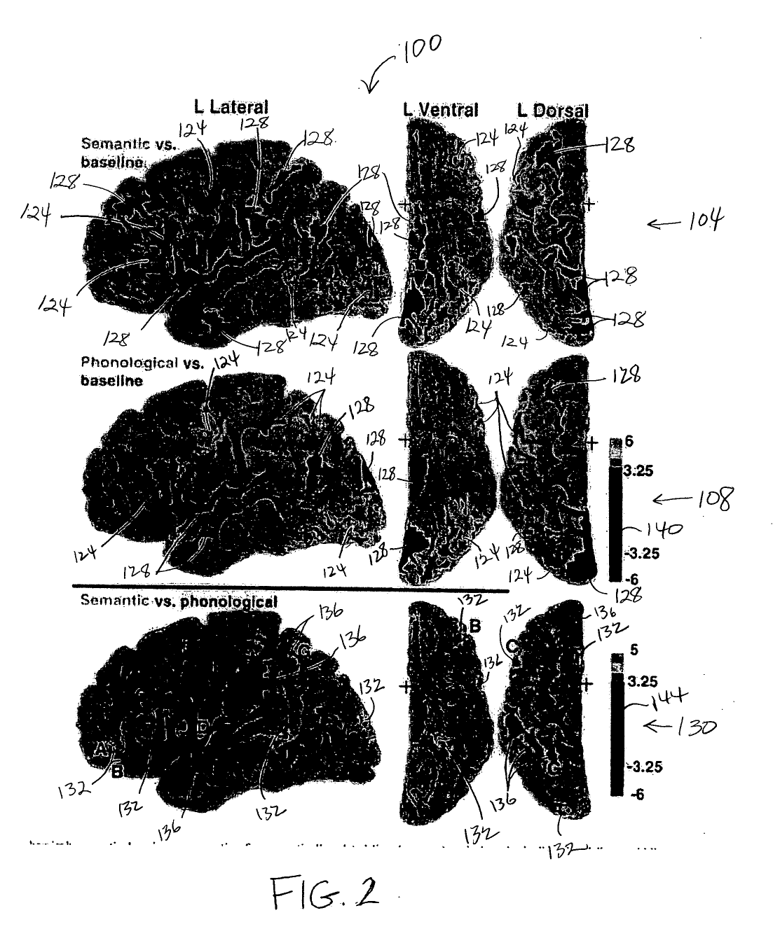

Various cortical regions, shown in FIGS. 2 through 5 and described further below, are illustrated in color in McDermott, K. B., Petersen, S. E., Watson, J. M., & Ojemann, J. G., A procedure for identifying regions preferentially activated by attention to semantic and phonological relations using functional magnetic resonance imaging, Neuropsychologia 41 (2003), 293-303. The colored illustrations included in the foregoing article are incorporated herein by reference. References herein to colored portions of cortical regions, and to various color bar indicators, are made with reference to the colored illustrations incorporated herein.

FIG. 2 shows left hemisphere cortical regions 100 more active for semantically-related lists (row 104) and phonologically-related lists (row 108) relative to the baseline activation state as determined by multiple-comparison corrected random-effects t-tests. For rows 104 and 108, regions 124 (shown in orange-yellow in the colored illustrations incorporated herein) are those showing greater activation for the task state than the baseline control state. Regions 128 (shown in blue) demonstrated greater activation during the baseline control period than during the task state. Row 130 exhibits regions 132 more active for semantically-related lists than phonologically-related lists (shown in orange-to-yellow) and regions 136 showing the opposite pattern (phonological>semantic, shown in blue). Regions of particular interest are labeled with letters, and corresponding peak coordinates can be seen in Tables 1 and 2 set forth below. Cases in which regions do not appear indicate regions occluded by more lateral cortical tissue. Labels in color bars 140 and 144 correspond to z-statistics (or level of statistical significance).

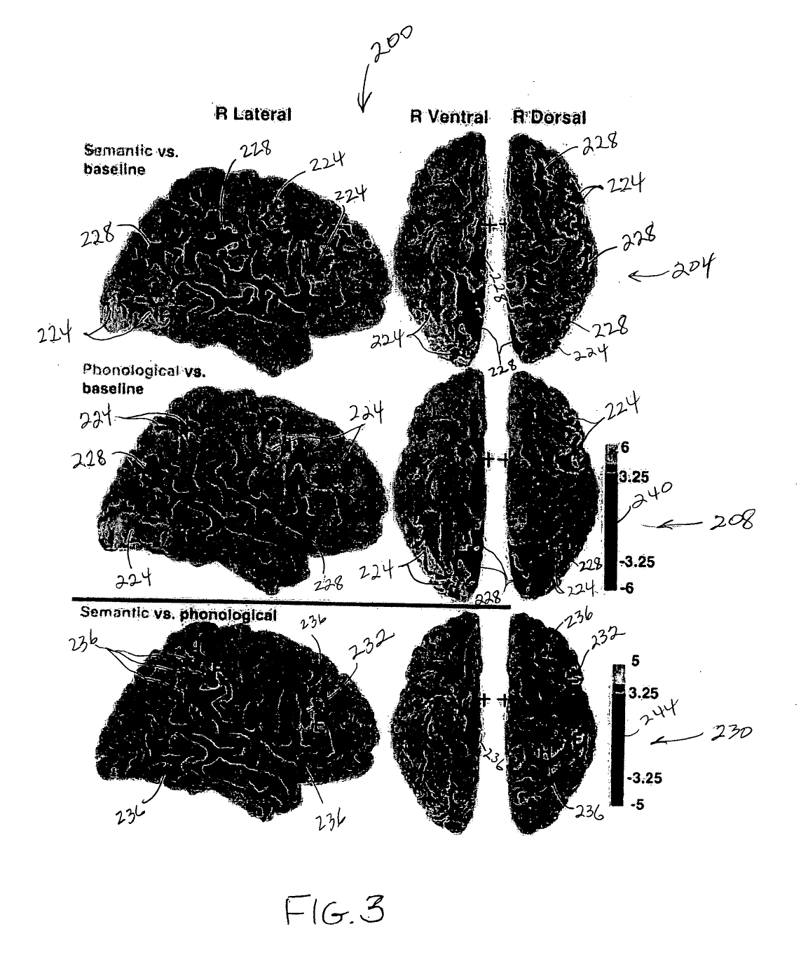

FIG. 3 shows right hemisphere cortical regions 200 more active for semantically-related lists (row 204) and phonologically-related lists (row 208) relative to the baseline activation state as determined by multiple-comparison corrected random-effects t-tests. For rows 204 and 208, regions 224 (shown in orange-yellow) are those showing greater activation for the task state than the baseline control state. Regions 228 (shown in blue) demonstrated greater activation during the baseline control period than during the task state. Row 230 exhibits regions 232 more active for semantically-related lists than phonologically-related lists (shown in orange-to-yellow) and regions 236 showing the opposite pattern (phonological>semantic, in blue). Regions of particular interest are labeled with letters, and the corresponding peak coordinates can be seen in Tables 1 and 2 below. Cases in which regions do not appear indicate regions occluded by more lateral cortical tissue. Labels in color bars 240 and 244 correspond to z-statistics (or level of statistical significance).

One can see from the top two rows of FIG. 2 (left hemisphere) and FIG. 3 (right hemisphere) that relative to a low-level baseline (fixating on a crosshair) semantic and phonological lists elicited activation in many of the same regions. The similarities highlight the point that the differences tend to represent differences in degree of activation within similar networks and not altogether different networks for semantic and phonological processing. Nonetheless, it is also evident from FIGS. 2 and 3 that activation in some regions was statistically significant in one task but not the other task.

Relative to the baseline control condition, both tasks activated left inferior frontal cortex (BA45/46 and BA44/45/46 extending into premotor and motor areas), right inferior frontal cortex (BA44/45), bilateral occipital cortex (BA17/18/19), bilateral fusiform gyrus (BA37), and (not shown in the figures) medial frontal gyrus (BA6, pre-supplementary motor area, pre-SMA), bilateral precuneus (BA7), and bilateral cerebellum. Although inferior frontal activations were strongly left-lateralized ventrally they became bilateral more dorsally and extended into right middle frontal gyrus.

In the top two rows of FIG. 2 it can be seen that relative to the low-level baseline condition, activation within left inferior frontal cortex was more extensive in the semantic condition than the phonological condition, especially in the anterior/ventral regions. Further, reliable left superior/middle temporal activation appears for the semantic condition but not the phonological condition.

Differences in activity for the two list types can be seen in row 130 of FIG. 2 (left hemisphere) and row 230 of FIG. 3 (right hemisphere). and in Tables 1 and 2 below. Whereas the blue activity at the top of the figures represents de-activation (of the active task state relative to the control state), it represents regions preferentially active for the phonological task (relative to the semantic task) in the bottom row, and the red-yellow represents regions preferentially active for the semantic task (relative to the phonological task). The activation magnitudes (% signal change) underlying these differences and the peak activation coordinates for the regions can be seen in Table 1 (semantic>phonological) and Table 2 (phonological>semantic).

TABLE 1
Regions demonstrating greater activation for lists of
semantically-related words than lists of
phonologically-related words

| | Coordinates | % Change** | | | Label |
|---|---|---|---|---|---|
| | (x, y, z) | Semantic | Phonological | Approximate location | FIGS. 2 & 3 |
| Frontal | −43, 39, 0 | 0.32* | 0.08* | Left inferior/middle frontal gyri (BA47) | A |
| | −37, 36, −12 | 0.32* | 0.07 | Left middle/inferior frontal gyrus (BA47/11) | B |
| | −37, 18, 18 | 0.52* | 0.24* | Left inferior frontal gyrus (BA44/45) | C |
| | −31, 3, 27 | 0.58* | 0.29* | Left inferior frontal gyrus (BA44) | C |
| | −34, 3, 51 | 0.28* | 0.06 | Left middle frontal gyrus (BA6) | |
| | 52, 27, 24 | 0.60* | 0.34* | Right middle/inferior frontal gyri | E |
| | | | | (BA46/44/9) | |
| | −7, 9, 54 | 0.30* | 0.08 | Medial frontal gyrus (pre-SMA, BA6) | |
| Temporal | −58, −45, 0 | 0.28* | 0.08 | Left middle/superior temporal gyrus | I |
| | | | | (BA22/21) | |
| Occipital | −16, −96, −3 | 0.30* | 0.19* | Left cuneus (BA17) | |
| | −19, −99, 12 | −0.11 | −0.36* | Left cuneus (BA18) | |
| Cerebellum | 19, −81, −33 | 0.15* | −0.02 | Right cerebellum | |
| | 31, −75, −36 | 0.06 | −0.09* | Right cerebellum | |
| | −10, −78, −33 | 0.16* | 0.04 | Left cerebellum | |
| Fusiform | −34, −45, −18 | 0.33* | 0.19* | Left fusiform gyrus (BA37) | |

Coordinates correspond to peak activations, magnitudes correspond to percent signal change relative to baseline, and asterisks (*) indicate activation magnitudes greater than baseline (fixation) levels (P < 0.05). Regions shown in bold font are those demonstrating activation in the positive direction for the semantic condition relative to baseline. ** Semantic > phonological

TABLE 2
Regions demonstrating greater activation for lists of phonologically-related words
than lists of semantically-related words

| | | | | | Label |
|---|---|---|---|---|---|
| | | % Change** | | | |
| | Coordinates | Semantic | Phonological | Approximate location | & 3 |
| Frontal | −55, 3, 15 | −0.06 | 0.14* | Left inferior frontal/precentral gyri (BA6/44) | D |
| | 40, 3, 0 | −0.15* | 0.04 | Right insula | |
| | 31, 24, 45 | −0.22* | −0.10* | Right middle frontal gyrus (BA8) | |
| Parietal | −43, −39, 36 | −0.21* | 0.07 | Left inferior parietal gyrus (BA40) | |
| | −55, −36, 30 | −0.10* | 0.04 | Left inferior parietal-supramarginal gyrus (BA40) | |
| | −58, −33, 39 | −0.16* | 0.07 | Left inferior parietal lobule (BA40) | |
| | −40, −42, 57 | −0.21* | 0.09 | Left inferior parietal lobule (BA40) | |
| | 43, −39, 45 | −0.05 | 0.10* | Right inferior parietal lobule (BA40) | F |
| | 52, −30, 27 | −0.19* | −0.10* | Right inferior parietal lobule (BA40) | |
| | 55, −33, 36 | −0.28* | −0.13* | Right inferior parietal lobule (BA40) | |
| | 52, −45, 30 | −0.20* | −0.06 | Right supramarginal gyrus (BA40) | |
| | −13, −69, 39 | −0.28* | −0.13* | Left precuneus (BA7) | |
| | 13, −60, 48 | −0.14* | 0.03 | Right precuneus (BA7) | |
| | 19, −69, 33 | −0.23* | −0.07 | Right precuneus (BA7) | |
| | −31, −57, 48 | 0.06 | 0.26* | Left superior/inferior parietal lobule (BA7/40) | G |
| | 31, −48, 51 | 0.03 | 0.21* | Right superior/inferior parietal lobule (BA7/40) | H |
| Occipital/temporal | −40, −81, 6 | 0.00 | 0.10 | Left middle occipital gyrus (BA19) | |
| | 43, −60, −6 | 0.35* | 0.49* | Right middle occipital gyrus (BA19) | |
| | 10, −69, 30 | −0.76* | −0.58* | Right cuneus/precuneus | |
| Cingulate | 4, −30, 39 | −0.28* | −0.12* | Right posterior cingulate (BA31) | |

Coordinates correspond to peak activations, magnitudes correspond to percent signal change relative to baseline, and asterisks (*) indicate activation magnitudes greater than baseline (fixation) levels (P < 0.05). Regions shown in bold font are those demonstrating activation in the positive direction for the phonological condition relative to baseline. ** Semantic < phonological

As can be seen by examining the orange-yellow regions in FIG. 2, preferential activation for semantic processing was observed in the LIFG both anteriorly/ventrally (BA47) and posteriorly/dorsally (BA44/45). In addition, regions within left superior/middle temporal gyrus (BA22/21), left occipital cortex (BA18/17), left fusiform gyrus (BA37), and right frontal cortex (BA9/46, shown in FIG. 3) showed this pattern of greater activation for semantic than phonological processing.

Preferential activation for phonological processing (shown in blue) occurred in left premotor cortex along the posterior border of the inferior frontal gyrus (BA6/44). In addition, regions within bilateral inferior parietal cortex (BA40) and precuneus (BA7) showed similar patterns.

Within frontal cortex, greater activation for semantic than phonological lists was observed within left anterior/ventral inferior frontal cortex (BA47; peak −43, 39, 0, labeled A in FIG. 2). A similarly left-lateralized activation pattern was seen in a region even further ventral (BA47/11; peak −37, 36, −12, labeled B in FIG. 2, best seen in the ventral view). In both cases there was reliable activation (relative to baseline) for the semantic lists but little (BA47) or not significant (BA47/11) activation for the phonological lists.

As can be seen in the region labeled C in FIG. 2, a separate region within left inferior frontal cortex, which is found dorsal and posterior to those just described, also showed preferential activation for semantic lists. For region definition this activation was separated into separate components (around the two peaks found by the search algorithm, −37, 18, 18; −31, 3, 27), although this was a large area of activation and may represent one large functional area. The activation spread along the IFG (BA44, along the border with BA45) and into middle frontal gyrus. Although greater activation was found for semantic lists, these regions showed robust activation for both semantic and phonological lists (all magnitudes reliably exceeded baseline magnitudes).

Posterior to this region within posterior/dorsal IFG was a functionally distinct region (labeled D), which demonstrated the opposite pattern: greater activation for the phonologically-related lists. This pattern was found along the border of the left precentral and inferior frontal gyri (peak −55, 3, 15, BA6/44) and extended ventrally into left insular cortex. This region demonstrated reliable activation (relative to baseline) for the phonological lists but not the semantic lists (see Table 2).

A single region in right frontal cortex showed greater activation for semantic than phonological processing (peak 52, 322 27, 24, labeled E in FIG. 3). Two right frontal regions demonstrated the opposite pattern (i.e. phonological>semantic); however, they demonstrated decreases in activity relative to baseline in the semantic condition but less negative activations (or nonsignificant activity) in the phonological conditions.

Whereas most of the left IFG differences involved a semantic preference, multiple regions in parietal cortex demonstrated a phonological preference. These included regions within bilateral inferior (BA40) parietal cortex in the vicinity of the supramarginal gyrus and bilateral precuneus (BA7).

Unlike the patterns seen throughout most of frontal cortex but similar to those frontal regions most recently discussed, many of the parietal regions demonstrated decreases in activity relative to baseline in the semantic condition but non-significant activations (or less negative activations) in the phonological conditions.

There were three parietal regions in Table 2 that demonstrated strong positive activation for the phonological task (peaks 43, −39, 45; −31, −57, 48; 31, −48, 51 for regions labeled F, G, and H, respectively).

A single peak within temporal cortex was obtained in the semantic-phonological t-test; specifically, a region in or near the superior temporal sulcus (BA22/21; I in FIG. 2) demonstrated preferential activation for the semantic lists (peak −58, −45, 0). Relative to baseline, this region exhibited reliable activation for the semantic lists but not the phonological lists.

Two regions in early visual areas demonstrated greater activation for semantically-related than phonologically-related lists (see Table 1). This might represent a manifestation of perceptual priming in unusually early visual regions. That is, the phonologically-related lists contained words that were orthographically similar (in addition to being phonologically similar). It may have been that reading words such as “weep”, “beep”, “heap” led to low-level priming of the visual system (relative to reading semantically-related words, which would be expected to show semantic priming but little or no low-level visual priming).

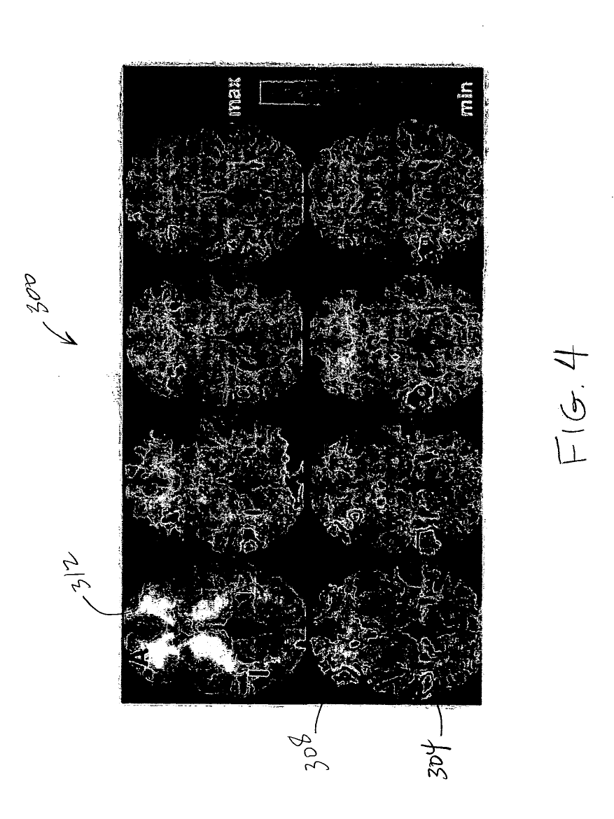

The strength of the manipulation performed in this experiment can be seen by examining the data of individual subjects. For many of the subjects, a simple contrast between activation levels for semantic and phonological lists revealed differences qualitatively similar to those seen at the group level. Most robust among these differences were the regions near the superior temporal sulcus (BA22/21) and left anterior/ventral IFG (BA47), which are highlighted with seven subjects' data referred to generally as 300 in FIG. 4. Contrasts between attention to semantics and to phonology at the individual subject level often revealed a similar region 304 in left superior/middle temporal gyrus (BA22/21) as being more active for semantically-related lists than phonologically-related lists. In addition, a region 308 in left inferior/middle frontal gyrus (BA47) can be seen. Upper left image 312 shows the region revealed by the multiple-comparison-corrected whole-brain random effects analysis (t-test) across all 20 subjects; A and I refer to region labels given in FIG. 2. For the seven individual subject images, increasing color intensity reflects increasing level of statistical significance.

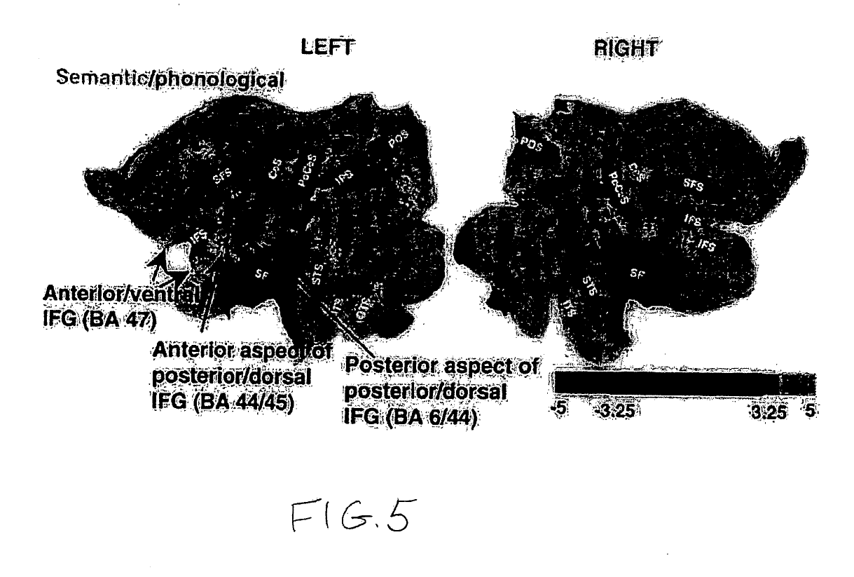

FIG. 5 displays the semantic-phonological t-test data (displayed in rows 130 and 230 of FIGS. 2 and 3) in flattened space. FIG. 5 shows two-dimensional, flattened representations of the cortical regions emerging from the semantic/phonological contrasts. An anterior/ventral region (BA47; labeled A in FIG. 2) showed preferential activation for semantic lists. Within more posterior frontal regions, there were further functional distinctions. A region in the more anterior aspect of posterior LIFG (BA44/45) showed preferential activation for semantic processing, whereas a more posterior region (BA6/44) demonstrated the opposite pattern. Major sulcal landmarks are labeled; abbreviations are superior frontal sulcus (SFS), inferior frontal sulcus (IFS), Sylvian Fissure (SF), central sulcus (CeS), postcentral sulcus (PoCeS), intraparietal sulcus (IPS), parieto-occipital sulcus (POS), superior temporal sulcus (STS), inferior temporal sulcus (ITS), and occipital-temporal sulcus (TOS).

One of the benefits of such a display is that the entire left and right hemisphere cortical activations can be viewed together. These projections can be used to highlight distinctions being made among frontal regions. As can be seen in FIG. 5, attention to semantics and to phonology can activate functionally separable regions within inferior frontal cortex. Regions within ventral/anterior IFG show greater activation to semantic than phonological lists. In addition, there are functionally distinct regions within posterior/dorsal IFG; the anterior aspect (BA44/45) shows preferential activation for semantic processing, whereas a more posterior region close (but not contiguous) to this region shows the opposite pattern: preferential activation to phonology (BA6/44).

In addition, the preferential activation in left superior/middle temporal cortex can be seen in FIG. 5, as can the single right hemisphere region showing preferential activation for attention to semantics (relative to phonology).

The results obtained here are consistent with a large body of neuroimaging of reading/language studies that demonstrate differential activation patterns for semantic and phonological processing within left inferior frontal cortex, left superior/middle temporal cortex, bilateral inferior parietal cortex, precuneus, left fusiform gyrus, and right cerebellum.

Both attention to semantic and to phonological processing activate a large swath of cortex along the IFG that (relative to a low-level baseline measure) appears somewhat similar. Notably, within dorsal/posterior IFG there appears to be an anterior/posterior distinction such that the anterior component (BA44/45) aligns with semantic processing and the posterior portion (BA6/44) aligns with phonological processing.

The foregoing example demonstrates a method that can be used to efficiently and cleanly identify language regions within a single group of subjects and within a subset of individual subjects. Attending to relations among associated words is a fairly natural task, one which can be performed by a wide variety of subject populations (including people with incompletely-developed language, e.g. children). Hence, embodiments of the present invention can be readily adapted for the study of cross-population differences in reading (for example, tracking the development of language in children, and, as another example, examining differences between dyslexic and normal readers). Embodiments of the present method also can be used for subject groups who cannot tolerate long scanning sessions.

Because interpretable data can be obtained within individual people in about an hour of functional scanning, the foregoing method can be useful with respect to pre-operative scanning. Neurosurgical procedures for patients (e.g. with intractable seizures or brain tumors) in left frontal and temporal cortices often require localization of language function. Contrasts that can be obtained using the foregoing method can identify language regions similar to those pinpointed by intraoperative cortical mapping. Semantic-phonological comparison and semantic and phonological lists together relative to baseline can be used, for example, to determine which contrasts predict localization of function within the operating room.

The above described lists can be designed to selectively challenge such systems as semantic and phonological systems, and this feature of the lists is one that is thought to give rise to false recall. Additionally, this naturally-occurring selective activation is enhanced by instructing people to attend to relations among words within lists. Subjects are presented with a cue (for example, “meaning” or “rhyme”) so that they would not need to figure out which dimension to attend to during presentation of the first several words. In addition, words are presented rapidly so as to challenge the systems of interest and to leave few cognitive resources for processing alternate dimensions of the words. That is, when presented, for example, with “bed”, “rest”, “awake”, etc. rapidly and told to attend to the semantic relations among the words, people cannot readily ponder the phonological characteristics of the words. Likewise, when presented, for example, with “beep”, “weep”, “peep”, etc. and asked to attend to the rhyming aspects of the words, people have little time to attend to the words' meanings. This feature is in contrast to tasks such as semantic generation and semantic decisions, which have led to substantial understanding of the neural bases of language but also are likely less process-pure in that their slow nature leaves time and resources for multiple confounding processes to intrude. Additionally, these methods make metalinguistic response demands that are not present in embodiments of the present method, in which no overt responding is required.

A rapidly-alternating blocked design sequence may be employed, which has been shown to be one of the most efficient, robust means of acquiring fMRI data. Additional embodiments can include longer or shorter word lists and/or other materials and/or other numbers of functional runs. Other word lengths and/or relationships among list words also are possible. Processing a word list at presentation rates described herein can be challenging to a subject, even when the subject only thinks about the words. Such challenge to a subject can be found also in embodiments in which duration of a presentation is increased and/or list words are simplified for use by some subject groups.

Regions of the brain involved in linguistic processing are robustly activated by this method, especially regions left inferior frontal and left superior/middle temporal cortices. This fact combined with the efficiency with which the data can be obtained (for example, in about one hour of functional scanning) suggests a wide range of possibilities for this technique. For example, embodiments of the present invention can be used in identifying language regions within individuals with brain tumors or epilepsy to aid neurosurgeons in surgical planning.

Attending to relations among associated words is a fairly natural task, one which can be performed by a wide variety of people at a wide range of intelligence levels. Embodiments can be practiced relative to adolescents and children, as well as people with low verbal IQ. In one embodiment, words are auditorily presented over headphones, for example, to a blind person. Embodiments of the present invention can be beneficial in assessing people who cannot tolerate long scanning sessions. In sum, embodiments of the present invention offer means for generating robust, interpretable data with respect to language function within individual people, without surgical intervention.

Nevertheless, in appropriate circumstances, embodiments can be practiced in coordination with surgical electrical stimulation mapping. Using the above method can increase the efficiency of electrical stimulation mapping, by suggesting to a surgeon which sites will likely be critical for language in a patient. Increasing the efficiency of electrical stimulation mapping is desirable because performing language tasks during surgery is effortful for the patient and is time consuming. In addition, data obtained using embodiments described herein can be invaluable in cases in which electrical stimulation mapping does not work well, for example, when swelling causes a patient to become aphasic during surgery and therefore unable to perform the language task needed for the surgeon to identify language regions intraoperatively. Scanning can be performed in about one hour using functional magnetic resonance imaging. In appropriate situations, embodiments can be practiced for preoperative assessment of patients awaiting neurosurgery, in place of intraoperative electrical stimulation mapping. Modifications can be made, for example, to word lists so as to be useful for assessing individuals speaking languages other than English.

Embodiments of the present method can produce cleaner, more robust data than most previous attempts at identifying language regions within individuals. This is especially true with respect to regions within left middle/superior temporal cortex, which are frequently important for surgical planning but have been difficult to identify within individuals using prior functional neuro-imaging techniques.

Embodiments of the present invention can result in robust, clean language maps, e.g., for patients who are awaiting neurosurgery and for pediatric patients. It can be seen from the foregoing description that embodiments of the present invention provide improvements over the more invasive technique of electrical stimulation mapping during surgery and also over the Wada technique.

The description of the invention is merely exemplary in nature and, thus, variations that do not depart from the gist of the invention are intended to be within the scope of the invention. Such variations are not to be regarded as a departure from the spirit and scope of the invention.

What is claimed is:

1. A method of identifying one or more language regions in the brain of a subject, the method comprising:
presenting to the subject one or more lists of related words to selectively challenge one or more language systems of the brain; and
scanning the brain while presenting the one or more lists.

2. The method of claim 1 wherein presenting to the subject comprises:
asking the subject to pay attention to relations among the words;
cueing the subject as to how the words of a list are related; and
presenting the words at a rate whereby the subject can comprehend the words but is challenged to pay attention to the relations.

3. The method of claim 2 wherein presenting the words comprises:
displaying a first word for 560 milliseconds;
waiting for 50 milliseconds;
displaying a second word for 560 milliseconds; and
waiting for 12.5 seconds before presenting another list.

4. The method of claim 1 wherein to selectively challenge one or more language systems comprises to selectively challenge at least one of a semantic system and a phonological system.

5. The method of claim 1 wherein presenting one or more lists comprises presenting at least one list of semantically related words.

6. The method of claim 1 wherein presenting one or more lists comprises presenting at least one list of phonologically related words.

7. The method of claim 1 wherein scanning comprises performing functional magnetic resonance imaging.

8. The method of claim 1 wherein presenting to the subject further comprises alternating one or more lists of semantically related words with one or more lists of phonologically related words.

9. The method of claim 1 wherein presenting to the subject comprises at least one of presenting auditorily and presenting visually.

10. A system for identifying one or more language regions in the brain of a subject, comprising:
one or more lists comprising related words configured to be presented comprehensibly but rapidly to the subject; and
a scanner for scanning the brain while the one or more lists are presented.

11. The system of claim 10 wherein the words of at least one of the one or more lists are related semantically.

12. The system of claim 10 wherein the words of at least one of the one or more lists are related phonologically.

13. The system of claim 10 wherein the words of a list are configured to challenge a language system of the brain.

14. The system of claim 10 wherein configured to be presented rapidly comprises configured to be presented at a rate that challenges the subject to pay attention to relations among the words.

15. The system of claim 10 wherein the scanner comprises a functional magnetic resonance imaging scanner.

16. A method of identifying one or more language regions in the brain of a subject, comprising:
presenting to the subject one or more lists of related words;
cueing the subject as to how the words are related;
presenting the words at a rate whereby the words are comprehensible but that challenges the subject to pay attention to relations among the words; and
recording activity in the brain while the subject processes the words.

17. The method of claim 16 further comprising designing the one or more lists to challenge at least one cortical language system.

18. The method of claim 16 wherein recording activity in the brain comprises performing a functional MRI scan.

19. The method of claim 18 further comprising beginning to present a list while beginning a repetition time of the scan.

20. The method of claim 18 further comprising:
projecting data from the scan onto a surface of a structural brain image; and
flattening the projected data for display.

21. The method of claim 16 wherein while the subject processes the words comprises while the subject says the words silently and thinks about similarity in sounds of the words.

22. A list for use in identifying a language region in the brain of a subject, the list comprising a plurality of related words configured to challenge a language system of the brain while being presented to the subject.

23. The list of claim 22 wherein the words are semantically related.

24. The list of claim 22 wherein the words are phonologically related.

25. The list of claim 22 wherein configured to challenge a language system comprises configured to challenge at least one of a semantic system and a phonological system.

25. The list of claim 22 configured for presentation to the subject at a rate relating to a repetition time of a scanner.

27. The list of claim 22 configured with one or more additional lists to selectively challenge one or more language systems of the brain while being presented to the subject.
